(19) United States
(12) Patent Application Publication
(10) Pub. No.: US 2022/0225902 A1
(43) Pub. Date: Jul. 21, 2022

(54) THERAPEUTIC TRANSDERMAL BIOREACTOR OR TRAP PATCH FOR DIABETES, PHENYLKETONURIA, AUTOIMMUNE, HYPERCHOLESTERINAEMIA AND OTHER DISORDERS

(71) Applicant: Vasileios LEKKOS, Essex (GB)

(72) Inventor: Vasileios LEKKOS, Essex (GB)

(21) Appl. No.: 17/614,393

(22) PCT Filed: Jun. 3, 2020
(86) PCT No.: PCT/IB2020/055221

§ 371 Date: Nov. 26, 2021

(30) Foreign Application Priority Data
Jun. 5, 2019 (GB) 1908043.1
Aug. 6, 2019 (GB) 1911263.0
Nov. 4, 2019 (GB) 1915959.9
Nov. 23, 2019 (GB) 1917094.3
Dec. 2, 2019 (GB) 1917532.2

(51) Int. Cl. A61B 5/145; A61B 5/00; A61M 1/34; A61M 1/36

(52) U.S. Cl. CPC A61B 5/14514; A61B 5/14532; A61B 5/685; A61M 1/34; A61M 1/3659; A61M 1/3687; A61M 2230/201; A61M 2205/3303; A61M 2205/3592; A61M 2205/3569; A61M 2205/3606; A61M 2205/8206; A61M 2205/106

(57) ABSTRACT

A transdermal patch comprising: a microneedle in a protruding position, protruding from the transdermal patch; and immobilised functional molecules; wherein a fluid path is provided between the distal tip of the microneedle and the immobilised functional molecules; and the functional molecules are selected to interact with selected target molecules so as to convert or trap said target molecules. The device intends to dynamically curb the postprandial insulin spike offering an unprecedented therapeutic effect for diabetes and obesity with significant benefits in morbidity and without strict diet. The device offers unprecedented phenylketonuria management that removes the burden of strict diet. The device offers easy removal of autoantibodies, low density lipoprotein and other pathogenic molecules. The device can be used in any disorder that requires enzymatic replacement or elimination of pathogenic molecules via biochemical conversion or trapping.

### FIELD OF THE INVENTION

The invention relates to a transdermal patch, and in particular a transdermal patch for therapeutic uses, including the reduction of postprandial glucose spikes in people suffering from diabetes.

### BACKGROUND OF THE INVENTION

Transdermal patches are known with a variety of functions, such as biosensing, drug delivery, and even the generation of useful energy. They are compact and convenient and cause only minimal interference in the lives of their wearers.

EP1512429 discloses a transdermal patch comprising a plurality of microneedles coated in a reservoir agent (for example, a sugar matrix) containing an active agent or drug to be delivered through the outer layer of skin into the body.

It is sometimes desirable to remove substances from the body, or convert them into other substances, rather than to deliver them into the body.

WO2015193624A1 discloses a reactor leading to the chemical transformation of a compound interacting with the reactor, that will for example constitute a glucose killer, for example by transforming glucose into a compound that will be eliminated by the body. This is one of the principles proposed by the present invention, but in the form of an implantable bioreactor with substantially different features.

### STATEMENT OF INVENTION

A first aspect of the invention provides a transdermal patch comprising: a microneedle in a protruding position, protruding from the transdermal patch; and immobilised functional molecules; wherein a fluid path is provided between the distal tip of the microneedle and the immobilised functional molecules; and the functional molecules are selected to interact with selected target molecules so as to convert or trap said target molecules.

The transdermal patch may further comprise an electromechanical actuator mechanism, controllable to cause the microneedle to extend to or retract from the protruding position.

The transdermal patch may further comprise an input from a sensor configured to detect a characteristic associated with the selected target molecules in a fluid, wherein the electromechanical actuator mechanism is controllable to cause the microneedle to extend or retract dependent on the sensor input.

The electromechanical actuator mechanism may be manually controllable by a user to cause the microneedle to extend or to retract.

The microneedle may protrude from the transdermal patch when in its protruding position such that, when in use, its distal tip is in fluid communication with the interstitial fluid of a user.

The microneedle may protrude from the transdermal patch when in its protruding position such that, when in use, its distal tip is in fluid communication with the capillary blood of the user.

The immobilised functional molecules may be held within the microneedle. The immobilised functional molecules may alternatively be held within a reactor chamber disposed within the transdermal patch.

The transdermal patch may further comprise a cartridge containing the immobilised functional molecules, the cartridge being removably inserted into the reactor chamber.

The sensor may be disposed within the transdermal patch, the sensor comprising a sensor microneedle protruding from the transdermal patch.

The target molecule may be glucose and the immobilised functional molecules may be one of: glucose oxidase; glucose dehydrogenase.

The transdermal patch may further comprise a semi-permeable membrane across the fluid communication channel between the external surface of the microneedle and the immobilised functional molecules, for preventing blood and immune cells or large proteins to flow, in use, from the distal tip of the microneedle to the immobilised functional molecules.

### BRIEF DESCRIPTION OF DRAWINGS

The invention will be described, by way of example only, by means of an exemplary embodiment, with reference to the FIGURE:

FIG. 1 depicts a transdermal patch according to an embodiment of the invention.

### DETAILED DESCRIPTION

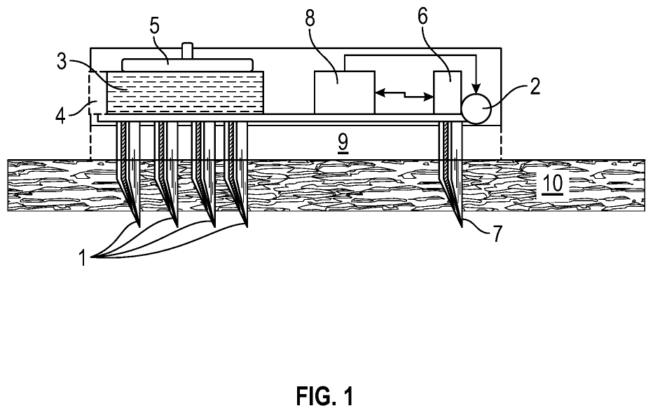

FIG. 1 depicts a transdermal patch according to the invention. In the depicted embodiment, the patch comprises a patch body, having a plurality of hollow microneedles 1 protruding from a first face.

The microneedles are optionally extendable and retractable by mechanism 2 which can be any standard mechanism for the extension and retraction of microneedles known in the art.

The mechanism may be activated by patient actuation or by other control procedures. Alternatively, the microneedles 1 may be permanently extended.

A chamber 3 is optionally provided, in fluid communication with the hollow microneedles 1. The fluid communication may be partial, for example mediated by a membrane or similar partial barrier (not shown). This typically surrounds the microneedles. The chamber may be surrounded by an oxygen permeable membrane, and the microneedles allow biological fluids to flow into the chamber, and at least the target molecules to diffuse into the chamber.

An access port 4 may be provided to the chamber 3, in order to replace the contents of the chamber 3, which will be discussed below.

A cooling device 5 may be provided in thermal communication with the chamber 3. This provides a heat sink for the chamber 3, and disperses the heat to the atmosphere outside the patch. As will become apparent, chemical reactions will occur in the chamber 3 when the device is in use, which will generate heat. The cooling device may be powered.

A biosensor 6 may be provided, having a biosensor microneedle 7. The biosensor microneedle 7 may be retractable and extendable by mechanism 2. Alternatively it may be permanently extended. Although this is depicted as part of the patch, it may alternatively be in a separate housing in remote communication with the patch. The biosensor is selected to detect characteristics associated with selected target molecules.

A controller 8 may be provided. This may be a microprocessor, or any device capable of processing instructions, receiving input data and outputting electronic commands.

The controller 8 may send instructions to the biosensor 6 to take a reading, and may receive reading results from the biosensor 6. It may instruct the microneedle extension and retraction mechanism 2 to extend or retract the microneedles 1 and/or the biosensor microneedle 7. The instruction to extend or retract the microneedles 1 may be dependent on the result of a reading from the biosensor 6.

In use, the transdermal patch is applied to an area of skin 10. The patch will stick to the skin by means of a suitable adhesive layer 9.

Although not shown in the FIGURE, a battery or other power source may be required. A wireless transceiver may be required for communication between different components of the system. For example, wireless communication may be necessary between the controller 8 and the biosensor 6. This may particularly be the case in embodiments in which the biosensor is separate to the transdermal patch.

The power sources, transceivers and sensors may be modular add-ons to the patch, through a suitable interface, so that they can be reused when the microneedles 1 and functional molecules are replaced.

It should be kept in mind that heat will be generated by the chemical reactions involved with the use of the transdermal patch of the invention. All materials should be selected to have suitable thermal properties. A larger number of microneedles 1 will increase the thermal safety of the device by increasing the total surface area through which the heat is transferred, thus minimising the heat flux and keeping the device within applicable safety regulations.

The purpose of the transdermal patch is to remove target molecules from biological fluid under the outer layer of skin. The biological fluid may, for example, be interstitial fluid. It may also or alternatively be capillary blood and/or venous blood. The length of the microneedles 1 can be carefully selected to target specific layers under the skin and specific biological fluids.

The removal of the target molecules may be either by capture or conversion. The target molecules may be bound or trapped by reaction with a selected functional molecule provided in the transdermal patch. Alternatively, the functional molecule may be selected to react with the target molecules in such a way as to result in a different molecule. This resultant molecule may then be returned to the user's body through the skin, where it will be excreted or otherwise disposed of by the body.

The functional molecules are held in the transdermal patch, for example by being immobilised in a suitable substance. The functional molecules may be held either inside the hollow microneedles 1, or in a chamber 3 in at least partial fluid communication with the hollow microneedles 1. In embodiments where the functional molecules are held within the hollow microneedles 1, it will be apparent that the chamber 3 may not be necessary and may be omitted from the patch.

Functional molecules may include one or more of: enzymes, apo-enzymes, antigens, antibodies, inorganic or organic catalysts, chelators, or other materials.

The functional molecules may be immobilised inside the patch (either in the chamber 3 or in the hollow microneedles 1) by means of a supporting material, selected to maximise the reactive surface area. The immobilisation may be by means of one of: bonding on surfaces with high specific surface area, porous materials, or nano-particles; entrapment in polymers, gels, hydrogels or other porous materials; the formation of aggregates; or the encapsulation of enzymes.

The supporting material may be electrically conductive, particularly when the target molecule is to be converted rather than captured. For example, redox polymers (a number of which, suitable for this purpose, have been developed), conductive nanoparticles, nanotubes, or porous materials such as carbon.

The hollow microneedles 1 may either have solid or perforated walls. They are open at the distal (skin-penetrating) end. If a chamber 3 is provided, they are also open at the proximal end. Otherwise, they are either open or perforated at the proximal end, so that oxygen can still diffuse from the atmosphere into the support material. A hydrogel, polymer, or similar substance is contained within the microneedles 1 and/or chamber 3, to facilitate diffusion (both of molecules from the biological fluid, and oxygen from the atmosphere). This substance should be selected for high thermal stability because of the heat generated by chemical reactions between the target molecules and the functional molecules.

A membrane or other semipermeable coating may be provided at the microneedles 1 or in the chamber 3. Typically the microneedles 1 are wholly coated in the coating. This may protect the device from immune reactions, or contact with large cells and proteins. It may also protect the body from the leakage of functional molecules.

In use, the patch is placed on a user's skin 10, and the biological fluid containing the target molecules will flow into the microneedles 1 when they penetrate the skin 10. Where a chamber 3 is provided, the biological fluid flows from the microneedles 1 into the chamber.

The target molecules diffuse to the immobilised functional molecules, and interact with them. The interaction may change the target molecules chemically, biochemically, physically or biologically. The interaction may alternatively bind the target molecules and keep them isolated from the body of the user.

An exemplary embodiment will be described for the treatment of diabetes. In particular, the transdermal patch of the invention can be used to curb a postprandial glucose and insulin spike.

Use of the device in this away may decrease insulin resistance in a user, and reduce the time in conditions of hyperglycemia and hyper insulinemia. It may also serve to remove calories taken from meals via the use of gluconolactone, providing further anti-diabetic and anti-obesity effects, and cardiovascular protection.

The device of this example converts excess glucose when glucose is detected by the biosensor 6 to be high. The biosensor 6 may be constantly measuring glucose levels, for example having the biosensor microneedle 7 permanently extended and penetrating the skin. Alternatively the biosensor 6 may be controlled to extend the biosensor microneedle 7 and take a glucose reading at selected times, either scheduled or in response to a user actuation. The microneedles 1 may be extended in response to the measurement of a spike or exceeded threshold in glucose levels. They may be retracted after a fixed period of time has elapsed, or after the glucose or insulin levels are detected to have reduced below a threshold. The controller 5 will be provided with the timer circuitry if required.

Alternatively, the microneedles 1 may be extended manually by a user. This may occur, for example, if the user knows that a glucose peak is taking place, or suspects it, for example having taken his or her own reading, or shortly after eating. The microneedles 1 could then be caused to retract automatically, for example after a fixed period of time has elapsed. The time period (as well as the size of the patch) could be determined according to the particular metabolic condition of the patient. Alternatively, the microneedles 1 could be retracted manually by the user.

If using the embodiment, discussed above, in which different units or sections of the patch are controlled to extend or retract their microneedles 1 independently, the glucose conversion rate can be finely controlled by protruding a selected number of microneedles associated with a selected amount of enzymatic hydrogel. For example, if half of the maximum conversion rate is required, only half of the microneedles need to be extended. This would be controlled by the glucose sensor measurements or the retraction timer. For example, one square centimetre of microneedles could be retracted every ten minutes.

In some embodiments, the patch is modular, in which case the patch is divided into smaller individual and independent patches, or units that are contained in the same patch. The microneedles 1 of every unit of the patch (for example, every square centimetre) will be extendable and retractable independently. In this way, the rate of conversion or trapping of the target molecules can be more finely controlled.

The device may be configured to convert glucose into gluconolactone. It may convert the glucose into other molecules, depending on the selected functional molecules. It may use glucose converting enzymes or other catalysts as functional molecules. For example, the functional molecules may comprise: glucose oxidase, together with catalase for the neutralisation of H₂O₂; dehydrogenase; or other enzymes.

The resultant molecules are then retuned to the body to be disposed of by the kidneys. One by-product may be water, which may partially evaporate from the system before being returned to the body.

Cofactors which may be needed will be co-immobilised and regenerated using various techniques documented in the literature, such as electrochemical regeneration or electroenzymatic regeneration.

For example, a PQQ/FAD-depended glucose dehydrogenase may be immobilised on a conductive material (such as a hydrogel, polymer, or nanoparticles), with or without electron transfer mediators (depending on the enzyme and the immobilization material, e.g. osmium complexes), where the electrons from the glucose after oxidation will be transferred into the supporting material. From there, they will be consumed by a substance co-immobilised on the same supporting material, such as laccase or bilirubin-oxygen oxidoreductase, to reduce the oxygen diffused into the supporting material from the atmosphere via the semipermeable barriers.

Other glucose converting enzymes can be used, such as glucose oxidase or inorganic/organic catalysts.

The components of the device are not included for the purpose of measuring voltage or current for sensor purposes, nor to generate voltage or current for electricity generation purposes. Consequently, there is no electrode, but rather there is only a conductive supporting material. The only purpose of this is the transfer of electrons from the glucose dehydrogenase cofactor to the laccase/bilirubin oxidase cofactor, and then to the diffused atmospheric oxygen, and convert it into water (together with the protons and electrons produced by the glucose oxidation) in order to regenerate the enzymes. A hydrogenase can also be co-immobilised to produce some hydrogen and decrease the dependency on oxygen.

The oxygen diffusion to the reaction point can be facilitated by using a highly porous material or particles with immobilized enzymes, so that the surface area will be maximized and thus the oxygen diffusion will be maximized.

The supporting material by which the enzymes and their cofactors will be immobilized will absorb the interstitial fluid, and will allow glucose to diffuse and at the same time allow the oxygen to be diffused through pores in the supporting material. In this way, both the catalytic surface in contact with the interstitial fluid and the oxygen diffusion surface from the atmosphere will be maximized and optimized.

Both the oxidizing/reducing enzymes will be immobilized on a conductive polymer or other material that will facilitate electron transfer, and the interstitial fluid will be absorbed onto that polymer/material while the oxygen will diffuse from the atmosphere, and will be reduced to form water that will diffuse back into the interstitial fluid via the microneedles, or overflow into a chamber on top of the electrode where it will evaporate from pores due to the heat generated by the reactions, while also protecting the user's skin from the heat.

The large area of the catalytic surface in contact with the interstitial fluid and the oxygen will result in a high rate of glucose conversion. Furthermore, the thin layer of interstitial fluid, as it is absorbed onto the catalytic surface, will allow rapid oxygen diffusion from the atmosphere. The polymer/hydrogel will allow both glucose and oxygen diffusion efficiently via its matter and/or pores. Alternatively, the laccase or bilirubin oxidase can be immobilized on the opposite/outer side of the supporting material and the glucose dehydrogenase can be immobilized in the inner side. Then the oxygen will diffuse from the atmosphere and react on the outer side of the supporting material. The electrons will be available in the supporting material of laccase/bilirubin oxidase from the glucose oxidation which occurs on the inner side of the supporting material where the dehydrogenase is immobilised. The protons generated on the inner side will diffuse into the outer side so that the oxygen is reduced to water.¹ ¹ For electrodes diffusing both protons and electrons, see for example: doi.org/10.1016/j.eurpolymj.2010.10.022.

The heat produced by the device through the chemical reactions will evaporate the water and also minimize the thermal impact of the device on the body. No separated electrodes or wiring are needed as the device is not required to generate current or voltage. The acidity produced by the glucose oxidation will be neutralized by the laccase and thus there will be no acidity impacting the human body.

Other enzymatic/inorganic/organic-catalytic cascades can be used. For example, the glucose can be converted into sorbitol and then sorbose with the relevant enzymes, to be excreted. Alternatively, the glucose can be converted into fructose and then to allulose by d-psicose 3-epimerase, which would be a safe and non-caloric ingredient that is excreted.

The device can alternatively operate in a transvascular mode. In such a mode, the microneedles 1 are always extended, and therefore always penetrating the user's skin 10. In such an embodiment, a rotating aperture will selectively isolate the functional molecules supported within the hollow microneedles 1 from the biological fluid in the user's skin 10, for example in response to a control signal or actuating action. An intraperitoneal device may also be used. In these embodiments, inorganic catalysts may be suitable for the conversion of glucose, such as Au/Pt or carbon.

An example of a conversion rate to be achieved in order to curb the postprandial glucose spike (and thus minimise hyperinsulinemia) could be a glucose conversion of 10 g per hour. This could be achieved, e.g. with 1 mg or even less of a glucose oxidation enzyme. The enzyme dispersed in the polymer/gel due to its porosity (or on the microparticles due to their high specific surface area) could achieve several m², which allows a very high rate of glucose molecule collision (e.g. several grams of glucose per second) so that the mass transport may not inhibit the device. The hydrogel with the co-immobilised enzymes can be fitted in 500 microneedles of 26 g or less (more if the heat flux of the device needs to be decreased) or it can be fitted in a thin layer inside the chamber above the microneedles. The 10 g conversion of glucose could release, for example, less than 1.5 Watt of heat (which is less than the accepted upper safety limit). The infusion rate of gluconolactone and water produced can be, for example, less than 20 mL/h, which is less than the generally accepted lowest acceptable subcutaneous infusion rate. The overall patch can be a few square centimetres or less, depending on the optimisation of the microneedles and other parameters.

The invention could also be used in the treatment of patients with alcohol addiction. For example, alcohol dehydrogenase (or another alcohol converting enzyme/catalyst) could be used to remove alcohol from the blood of patients, gradually weaning them off alcohol. An alcohol biosensor could optionally be used for this purpose.

The device could also have a phenylalanine converting enzyme, such as phenylalanine ammonia-lyase, dehydrogenase, hydroxylase, aminomutase, decarboxylase, transaminase, monooxygenase etc or other catalyst, to convert excessive phenylalanine in phenylketonuria patients. In one embodiment, the device could use phenylalanine aminomutase (D-beta-phenylalinine forming), to convert excess L-phenylalanine into D-beta-phenylalanine, which is less toxic than L-phenylalanine and protects against the toxicity of the same. Such a device could operate with or without biosensor-feedback control of the conversion function.

Similarly, the device could be used with urate and uricase for the treatment of uricemia. Electron accepting enzymes (or inorganic catalysts) such as laccase could be employed to transfer the electrons to the oxygen. In this way, the device could provide enzyme replacement therapy.

Triacylglycerol lipase could also be used with the device, to convert excess triglycerides in the body.

The device could be used with other enzymes or catalysts, to perform enzymatic functions for patients, as a form of enzyme replacement therapeutic intervention in metabolic disorders with impaired enzyme function.

The device can be used as an antibody/antigen trap. The biosensor 6 and microneedle extension and retraction mechanism 2 would not be necessary in such an embodiment. The functional molecules of the device would be immobilised antibodies or antigens, that will bind and trap their respective antigens and antibodies diffused from the biological fluid such that, for example, a pathogenic antibody or autoantibody will be removed from the body by way of a gradual and continuous plasmapheresis-like intervention, for therapeutic purposes for immune-related diseases or other diseases (for example, the removal of low-density lipoprotein using immobilised anti-LDL antibodies for treating hypercholisterinemia). A coating or membrane to prevent immune or other cells interacting with the functional surface of the patch will be needed. Once the patch is saturated, it will be replaced.

The device intends to dynamically curb the postprandial insulin spike offering an unprecedented therapeutic effect for diabetes and obesity with significant benefits in morbidity and without strict diet. The device offers unprecedented phenylketonuria management that removes the burden of strict diet. The device offers easy removal of autoantibodies, low density lipoprotein and other pathogenic molecules. The device can be used in any disorder that requires enzymatic replacement or elimination of pathogenic molecules via biochemical conversion or trapping.

It will be appreciated that many of the individual features of the embodiments described above are known in some form or other. The skilled person will therefore be able to construct the invention based on the present disclosure without the need for more than routine trial and error. For example, immobilisation techniques using the functional molecules and support materials discussed above have been used in the prior art. Biosensors with closed loop feedback are also known in similar devices. Extension and retraction mechanisms for microneedles are known. Nevertheless, where these features are known in the prior art, it is in service to different functions, such as drug delivery, biosensing, and/or biofuel cells for energy generation. The novelty of the present invention lies in the combination and scale of the features and the purpose in service to which they have been combined and adapted.

Although the invention has been described with reference to one or more preferred embodiments, the embodiments described and depicted are not intended to limit the scope of the invention. The scope of the invention is limited by the claims.

1. A transdermal patch comprising:
a microneedle in a protruding position, protruding from the transdermal patch; and
immobilised functional molecules; wherein
a fluid path is provided between the distal tip of the microneedle and the immobilised functional molecules; and
the functional molecules are selected to interact with selected target molecules so as to convert or trap said target molecules.

2. A transdermal patch according to claim 1 further comprising an electromechanical actuator mechanism, controllable to cause the microneedle to extend to or retract from the protruding position.

3. A transdermal patch according to claim 2 further comprising an input from a sensor configured to detect a characteristic associated with the selected target molecules in a fluid, wherein the electromechanical actuator mechanism is controllable to cause the microneedle to extend or retract dependent on the sensor input.

4. A transdermal patch according to claim 2 wherein the electromechanical actuator mechanism is manually controllable by a user to cause the microneedle to extend or to retract.

5. A transdermal patch according to any preceding claim wherein the microneedle protrudes from the transdermal patch when in its protruding position such that, when in use, its distal tip is in fluid communication with the interstitial fluid of a user.

6. A transdermal patch according to any one of claims 1 to 4 wherein the microneedle protrudes from the transdermal patch when in its protruding position such that, when in use, its distal tip is in fluid communication with the capillary blood of the user.

7. A transdermal patch according to any preceding claim wherein the immobilised functional molecules are held within the microneedle.

8. A transdermal patch according to any one of claims 1 to 6 wherein the immobilised functional molecules are held within a reactor chamber disposed within the transdermal patch.

9. A transdermal patch according to claim 8 further comprising a cartridge containing the immobilised functional molecules, the cartridge being removably inserted into the reactor chamber.

10. A transdermal patch according to claim 3 wherein the sensor is disposed within the transdermal patch, the sensor comprising a sensor microneedle protruding from the transdermal patch.

11. A transdermal patch according to any preceding claim wherein the target molecule is glucose and the immobilised functional molecules are one of: glucose oxidase; glucose dehydrogenase.

12. A transdermal patch according to any preceding claim further comprising a semi-permeable membrane across the fluid communication channel between the distal tip of the microneedle and the immobilised functional molecules, for preventing blood and immune cells or large proteins to flow, in use, from the distal tip of the microneedle to the immobilised functional molecules.
